(19) United States
(12) Patent Application Publication
(10) Pub. No.: US 2025/0231106 A1
(43) Pub. Date: Jul. 17, 2025

(54) MULTI-CANCER DETECTION

(71) Applicant: DXCOVER LIMITED, Glasgow (GB)

(72) Inventors: Matthew J BAKER, Glasgow (GB); James CAMERON, Glasgow (GB); David PALMER, Glasgow (GB)

(21) Appl. No.: 18/844,719

(22) PCT Filed: Mar. 8, 2023
(86) PCT No.: PCT/GB2023/050544

§ 371 Date: Sep. 6, 2024

(30) Foreign Application Priority Data
Mar. 9, 2022 (GB) 2203260.1

(51) Int. Cl. G01N 21/552; G01N 21/35; G01N 21/3577; G01N 33/49

(52) U.S. Cl. CPC G01N 21/552; G01N 21/3577; G01N 33/49; G01N 2021/3595; G01N 2201/1296

(57) ABSTRACT

A method of detecting whether or not a subject has a cancer irrespective of the stage or type of cancer comprises performing an IR spectroscopic analysis comprising wavelengths between 400 4000 cm⁻¹ on a blood sample from the subject, to produce a spectroscopic signature characteristic of the blood sample, wherein said spectroscopic signature of the blood sample is analysed against representative signatures from previous subjects with and without cancer, wherein the subjects with cancer comprise subjects with different types of cancer and different stages of cancer, in order to detect whether or not the subject has a cancer, based upon the spectroscopic signature obtained from the subject.

### FIELD

The present disclosure relates to methods of detecting cancer using spectroscopic technology.

### BACKGROUND

Early detection of cancer is vital to improve patient prognosis and reduce mortality rates. Cancer killed 10 million people in 2020, representing one of the leading causes of death worldwide (1). An early diagnosis can inhibit progression of the disease before the tumor proliferates to a more advanced stage. Thus, with earlier detection many patients may be cured with surgical intervention alone, meaning they would not have to endure aggressive systemic treatment like radiotherapy or chemotherapy—a reported 70% of early stage tumors (stage I) are treated with surgery, whereas only ˜13% of stage IV cancers undergo surgical resection (2). If the disease is not identified quickly, and the cancerous neoplasm is allowed to grow and metastasize to a distant secondary site, then the outlook for the patient is often dismal, as current therapeutic methods are rarely effective for late-stage disease (3). Current screening methods tend to focus on one single target organ, such as breast and colorectal cancers. This approach is not currently economically viable for many cancer types, and in fact over two-thirds of lethal cancers do not have any active screening options, making earlier detection extremely difficult (4).

There has recently been a plethora of research studies into liquid biopsy technologies, which have the potential to transform cancer diagnostics (5). Many of these tests are based upon genomic methods, which utilize genetic material such as circulating tumor DNA (ctDNA) and/or cell-free DNA (cfDNA). Remarkably, there are approximately 150,000 scientific papers that document thousands of biomarkers with apparent clinical utility, yet only 1% of known markers are routinely used in clinical applications (6). There are only a few commercialised liquid biopsies currently available, which are mainly targeted at single-cancer detection. For example, the SelectMDx test for prostate cancer is a urine-based test and has been successfully launched in the USA and Europe by MDxHealth (7). The ExoDx test (Exosome Diagnostics), also for prostate cancer, provides an individual risk score for the patient which determines whether they should be referred for biopsy (8). The commercial use of these single-cancer liquid biopsies evidences the potential of early detection strategies within healthcare systems.

A promising method of cancer detection using liquid biopsies is based on spectroscopic signatures obtained using attenuated total reflection infrared (IR) spectroscopy to discern cancer vs non-cancer in a subject (see WO2017/221027; Baker et a)). However, this method does not address a need in the art for the development of methods capable of detecting a general cancerous signature to determine cancerous status covering multiple cancers, including the type of cancer in a subject and for example, detecting early-stage cancers. Thus, the development of a robust test capable of detecting multiple cancer types would be transformational within the diagnostics field. Greater than 70% of cancer deaths occur in low- and middle-income countries (9), so there is a compelling need for development of low-cost, highly sensitive multi-cancer tests.

It is amongst the objectives of the present disclosure to develop an early detection diagnostic system that would mitigate one or more of the aforementioned disadvantages of existing screening methods.

### SUMMARY

The present disclosure is based in part on studies using infrared (IR) spectroscopy based methods to identify proliferative disorders, such as cancer, using liquid blood samples. This method is unique as rather than searching for individual biomarkers, it probes a wide range of biological features and produces a distinctive signature which represents a significant part or whole biochemical profile of the sample. The distinctive spectroscopic signature contains molecular information from the tumour as well as a host response, such as an immune response. The IR spectroscopy method may employ attenuated total reflection—Fourier transform infrared (ATR-FTIR) spectroscopy, which is particularly well-suited for the clinic as the methodology is minimally-invasive, cost-effective, little or no sample preparation is required, and reproducible results can be generated in a matter of minutes since analysis is rapid.

In a first aspect, there is provided a method of detecting whether or not a subject has a cancer irrespective of the stage or type of cancer, the method comprising:
performing an IR (such as ATR-IR) spectroscopic analysis comprising wavelengths between 400-4000 cm⁻¹ on a blood sample from the subject, to produce a spectroscopic signature characteristic of the blood sample, wherein said spectroscopic signature of the blood sample is analysed against representative signatures from previous subjects with and without cancer, wherein the previous subjects with cancer comprise subjects with different types of cancer and different stages of cancer, in order to detect whether or not the subject has a cancer, based upon the spectroscopic signature obtained from the subject.

Thus, the term “subject” herein refers to an individual on which detection is carried out, whereas the term “previous subjects” refers to individuals from which reference spectroscopic signatures, e.g. a database or dataset of reference spectroscopic signatures, are obtained and which can be used in subsequent comparison and/or correlation with a spectroscopic signature of the blood sample of the “subject” in order to carry out the detection, as explained below in more detail.

The term “blood” as used herein refers to whole blood or a fraction thereof. In one embodiment, the sample from the subject for the IR analysis is obtained from whole blood or a fraction thereof, such as serum or plasma. In one embodiment, the sample from the subject for the IR analysis is serum.

The term “cancer” refers to the physiological condition where cells exhibit abnormal and unregulated growth. “Cancer” as used herein, unless otherwise specified, generally refers to any type of cancer irrespective of the stage of cancer. Examples of cancer include, but are not limited to, bile duct cancer, bladder cancer, brain and central nervous system cancer, breast cancer, cervical cancer, colorectal cancer, kidney cancer, gallbladder cancer, leukaemia, liver cancer, lung cancer, stomach cancer, Hodgkin and non-Hodgkin lymphoma, melanoma, multiple myeloma, mesothelioma, osteosarcoma, oral (including lip and salivary glands) cancer, laryngeal and oro-, naso-, hypopharyngeal cancer, ovarian cancer, pancreatic cancer, prostate cancer, sarcoma, thyroid cancer, uterine and vaginal cancer, and testicular and penis cancer. In an embodiment, the cancer is selected from the group consisting of brain cancer, breast cancer, colorectal cancer, kidney cancer, lung cancer, ovarian cancer, pancreatic cancer and prostate cancer.

As used herein, “stage of cancer” refers to the size of the tumour and indication of the spread of the cancer from the tissue where the cancer has originated from, which enables clinicians to assess cancer progression. Most cancers that involve a tumour can be categorised into four stages, according to the Overall Stage Grouping, also known as Roman Numeral Staging. Stage I refers to when the cancer is small and localised within the organ the cancer started in. Stage II refers to when the cancer has grown and is larger than stage I but has not spread to surrounding tissues. In some types of cancers, stage II may include instances where the cancer has spread to nearby lymph nodes. Early-stage cancer typically refers to stage I or stage II cancers. Stage III refers to when the cancer is larger and may have spread to the surrounding tissues, such as nearby tissues and/or lymph nodes. Stage IV refers to when the cancer has spread to other organs or has advanced to a substantial volume for organ confined tumours (e.g. brain cancer) (also referred to as advanced or metastatic cancer). In an embodiment the methods as described herein are particularly suited to the detection of early-stage cancers, typically stages I-III, or I-II. In another embodiment the methods as described herein are particularly suited to the detection of cancers irrespective of the stage of cancer, and/or the methods as described herein are particularly suited to the detection of cancers in any of stages I-IV.

As used herein, the term “detecting” is used broadly and may be used in terms of facilitating with a diagnosis and/or prognosis of a subject. The detection of cancer in a subject may comprise conducting an analysis against representative signatures obtained from (the) previous subjects. This may involve comparing and/or correlating the spectroscopic signature of the blood sample (or component thereof) to one or more reference spectroscopic signatures previously obtained by statistical analysis from (the) previous subjects with and without cancer and different stages of cancer. Comparing and/or correlating may be carried out using a suitable computational process or program. The method may comprise comparing and/or correlating, using a machine learning or deep learning process.

The machine learning or deep learning process may be or may comprise a neural network. The neural network may be or may comprise a convolutional neural network or a recurrent neural network. The convolutional neural network may comprise one or more of: at least one 1-dimensional convolutional layer, at least one down sampling layer and/or at least one batch normalization layer. The recurrent neural network may comprise one or more long-short term memory (LSTM) layers. Other exemplary algorithms which may be used include: (oblique) random forest with partial least squares (PLS), logistic regression, support vector machines or other machine learning algorithms at each node in each decision tree; other linear models such as LDA. The machine learning or deep learning models may be single models or ensembles of multiple models. Ensemble models may be built on different samples of the data using one or more of bootstrap aggregating (“bagging”) or random sampling.

The data may be, may comprise, may be derived from, or may be representative of directly measurable spectroscopic data, comprising or resulting from spectral measurements taken from the blood sample. The data may be or may comprise measurements, which are taken across the defined wavelength region, or may be or may comprise measurements obtained from two or more sub-regions, as will be discussed further herein.

According to an example of the present disclosure there is provided a computer readable medium carrying a computer program comprising computer readable instructions configured to cause a computer to carry out a method as described herein.

According to an example of the present disclosure there is provided a computer apparatus, such as embodied within, or connected to a spectrometer, comprising or configured to access: a memory storing processor readable instructions, and a processor arranged to read and execute instructions stored in said memory, wherein said processor readable instructions comprise instructions arranged to control the computer to carry out a method as described herein.

Examples of the subject matter and the functional operations described in this specification can be implemented in digital electronic circuitry, in tangibly-embodied computer software or firmware, in computer hardware, including the structures disclosed herein and their structural equivalents, or in combinations of one or more of them. Examples of the subject matter described in this specification can be implemented as one or more computer programs, i.e., one or more modules of computer program instructions encoded on a tangible non-transitory program carrier for execution by, or to control the operation of, data processing apparatus. Alternatively, or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. The computer storage medium can be a machine-readable storage device, a machine-readable storage substrate, a random or serial access memory device, or a combination of one or more of them.

The term “data processing apparatus” encompasses all kinds of apparatus, devices, and machines for processing data, including by way of example a programmable processor, a computer, or multiple processors or computers. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, or a combination of one or more of them.

A computer program (which may also be referred to or described as a program, software, a software application, a module, a software module, a script, or code) can be written in any form of programming language, including compiled or interpreted languages, or declarative or procedural languages, and it can be deployed in any form, including as a standalone program or as a module, component, subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, subprograms, or portions of code. A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Computers suitable for the execution of a computer program include, by way of example, can be based on general or special purpose microprocessors or both, or any other kind of central processing unit. Generally, a central processing unit will receive instructions and data from a read only memory or a random-access memory or both. The essential elements of a computer are a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, examples of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the internet.

The computing system can include clients and servers. A client and server are generally remote from each other and typically interact through a communication network. The relationship of client and server arises by virtue of computer programs running on the respective computers and having a client-server relationship to each other.

References are made herein to condition parameter curves, but it will be appreciated that such references are to the evolution of the degradation parameter and may comprise linear and/or arcuate sections such as sections that can be described by polynomial, exponential, power and other functions, and may be smoothly or gradually varying or may comprise sharp, angular transitions between regions.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination.

Moreover, although features may be described above as acting in certain combinations and even initially claimed as such, one or more features from a claimed combination can in some cases be excised from the combination, and the claimed combination may be directed to a subcombination or variation of a subcombination.

The methods of the present disclosure generally use infrared (IR) spectroscopic analysis. Various IR techniques known in the art may be employed in the methods of this disclosure. One method may use Fourier transform IR (FTIR) spectroscopic analysis. In FTIR, the IR spectra may be collected in the region of 400-4000 wavenumbers (cm⁻¹). Generally, the IR spectra may have a resolution of 10 cm⁻¹ or less, 5 cm⁻¹ or less, or approximately 4 cm⁻¹. The FTIR spectroscopic analysis may employ at least 10 scans, at least 15, or at least 30 scans. The FTIR spectroscopic analysis may employ at most 100 scans, at most 50 scans, or at most 40 scans. For example, 16 scans may be used. The scans may be co-added. As will be appreciated by the skilled person, the number of scans may be selected to optimize data content and data-acquisition time.

Prior to spectroscopic analysis, a background spectrum may be obtained. Such background spectra may provide correction for a background environment. For example, the background spectrum of air may be obtained to provide an atmospheric correction. Alternatively, or additionally, the background spectrum of a solution may be obtained, e.g. an aqueous solution such as phosphate buffered saline (PBS). In one embodiment, a background spectrum is obtained in order to provide for correction for a background environment. In one embodiment, the spectroscopic analysis may further comprise normalisation (e.g. standard normal variate), noise reduction (e.g. principal component analysis (PCA)-based) and derivatisation (e.g. 1st or 2nd) as pre-processing steps and/or Fourier transform IR spectroscopic analysis.

The claimed methods use IR spectroscopic analysis, such as Attenuated Total Reflection (ATR)-IR spectroscopic analysis, using blood samples from subjects. In some embodiments, the spectroscopic analysis used to obtain spectroscopic signature characteristic of the blood sample may be ATR-FTIR.

During a typical ATR-IR spectroscopic analysis, the blood sample of a subject may be loaded onto an internal reflection element (IRE) and IR light may travel through the IRE, and reflect (e.g. via total internal reflection) at least once off an internal surface of the IRE that is in contact with the sample. Such reflection may form an “evanescent wave” which penetrates into the blood sample to an extent depending on the wavelength of light, the angle of incidence and the indices of refraction for the IRE and the blood sample itself. The depth of penetration and path of reflected light can be altered by varying the angle of incidence and/or wavelength of incident light. The beam may be received by an IR detector as it exits the internal reflection element. The IRE is generally an optical material with a higher refractive index than the blood sample to enable the evanescent wave effect.

The IR spectroscopic signature characteristic of the blood sample (which may be referred to as the signature or fingerprint region) may typically be part or all of the relevant IR spectrum between 400 to 4000 cm⁻¹.

The spectroscopic signature characteristic of the blood sample is compared to a database of representative signatures previously obtained from samples from previous subjects with cancer or healthy subjects, in order to detect, through comparison of the respective signatures, whether or not the subject has cancer. The database comprises representative signatures from previous subjects with different types of cancer and different stages of cancer. Furthermore, the spectroscopic signature of the blood sample may be compared to a database of representative signatures obtained from samples from previous subjects with specific types of cancers to diagnose the type of cancer. Such comparison may be carried out using pattern recognition software and/or machine learning analysis known in the art and/or as described herein.

In one embodiment, the analysis against representative signatures from previous subjects comprises applying a trained model to the spectroscopic signature. The trained model may comprise a trained machine learning model, and optionally a neural network, a supporting vector machine (SVM) or a random forest (RF) decision tree. The trained model may comprise or function as a classifier by applying the or a probability threshold to a probability value output by the trained model. The method may additionally comprise selecting and/or varying the probability threshold thereby selecting and/or varying the specificity and/or sensitivity of the spectroscopic signature analysis. Further, the method may involve selecting the probability threshold based on a receiver operating characteristic (ROC) curve for the trained model, and/or selecting the trained model from a set of trained models based on ROCs for the set of trained models, for example thereby to obtain a desired specificity and/or sensitivity.

The term “sensitivity” is herein understood as the proportion of diseased subjects who are correctly identified as “positive” by a test. Thus, sensitivity can be defined as the percentage of true positives, as predicted by the test. A test with 100% sensitivity would correctly detect all patients who have a given disease. In other words, high sensitivity means a low occurrence of false negatives.

The term “specificity” is herein understood as the proportion of non-diseased subjects who are correctly identified as “negative” by a test. Thus, specificity can be defined as the percentage of true negatives, as predicted by the test. A test with 100% specificity would correctly detect all patients who do not have a given disease. In other words, high specificity means a low occurrence of false positives.

An advantageous feature of the methods described herein is the ability to detect cancer in a patient irrespective of the stage of cancer. In one embodiment, the methods are capable of detecting early-stage cancer, as an early medical intervention may increase the likelihood of patient survival. For this to be possible as a first-line diagnostic test, high sensitivity of the test is essential in order to ensure that all patients with cancer are correctly identified. Existing genomic cancer diagnostic tests, such as next-generation sequencing (NGS)-based ctDNA liquid biopsy tests, and methylation-based tests, are not sensitive enough to detect early-stage cancers. The spectroscopic liquid biopsy methods as disclosed herein differ from other tests as the probability threshold of the machine learning analysis can be adjusted to maximise either the sensitivity or specificity depending on clinical requirements. In one embodiment, there is provided a method comprising a spectroscopic liquid biopsy with machine learning analysis, wherein the probability threshold of the analysis is adjusted to maximise the sensitivity for detection of cancer, irrespective of the stage of cancer, using blood samples from patients. In a particular embodiment, the liquid biopsy based spectroscopic analysis can differentiate patients with early-stage cancer, such as stage I or stage II cancer, from subjects without cancer by maximising the sensitivity of the analysis.

In some application of diagnostic analysis, such as when stratifying subjects with cancer from those without cancer in a triage setting, classifiers with a high sensitivity and modest to low specificity may be more desirable than models with low sensitivity and high specificity. In other diagnostic applications, such as for general cancer detection with a population level screening test, classifiers with high specificity and modest to low sensitivity may be more desirable. The desired level of performance is generally selected based upon a trade-off that must be made between the number of false positive and false negatives that can each be tolerated for the particular diagnostic applications. Such trade-offs generally depend on the medical consequences of an error. In one embodiment, the method as described herein comprises adjusting the probability threshold of the machine learning algorithm to determine the sensitivity and/or specificity of the spectroscopic analysis.

A high sensitivity or specificity typically refers to a value greater than 70%. A modest sensitivity or specificity may typically be greater than 50% but less than 70%. In one embodiment, the sensitivity of the analysis is greater than 70% and specificity of the analysis is greater than 40%. In another embodiment, the sensitivity of the analysis is greater than 40% and the specificity of the analysis is greater than 70%. In one embodiment, the sensitivity of the analysis is at least 80% and specificity of analysis is at least 40%. In one embodiment, the sensitivity of the analysis is at least 40% and specificity of analysis is at least 80%. In certain embodiments, the sensitivity is at least 80% and specificity is at least 40%, 45%, 50%, 55%, 60%, 65%, 70% or 75%. In certain embodiments, the specificity is at least 80% and sensitivity is at least 40%, 45%, 50%, 55%, 60%, 65%, 70% or 75%.

Subsequent to identification of patients with cancer, the multi-cancer diagnostic method of the present disclosure can be adjusted to maximise the specificity in order to correctly identify the type of cancer of the patient. The ability to adjust the sensitivity and/or specificity in this manner highlights the potential of the method disclosed herein as a cost-effective test to triage patients in the clinic and enable translation to fit the current cancer pathway as some cancers require high sensitivity whereas others require high specificity for a first-line triage depending upon the next steps.

In one embodiment, the spectroscopic analysis of the present disclosure comprises conducting the spectroscopic analysis with high sensitivity to identify patients with cancer and subsequently conducting the spectroscopic analysis with high specificity to identify the patients without cancer.

In an alternative embodiment, the method as described herein may be adjusted to high sensitivity to identify patients with cancer and subsequently the patients with cancer may be directed to alternative tests for further analysis, such as other blood-based tests, NGS-based tests, protein markers and/or medical imaging (e.g. CT (computed tomography) magnetic resonance imaging (MRI), ultrasound, colonoscopy).

The present disclosure also provides a method of using the spectroscopic signature of the blood sample comprising specific molecular vibrational modes and/or detection of peaks at specific wavenumber regions to detect cancers. The specific molecular vibrational modes and detection of peaks at specific wavenumber regions of the spectroscopic signature may also be used to discern the type of cancer in patients with cancer. Detection of peaks at specific wavenumber regions are associated with molecular vibrational modes that may be used to identify the type of cancer in the subject irrespective of the stage of cancer.

In one embodiment, the method as described herein may be used to provide an indication of the type of cancer in a subject, such as brain cancer, breast cancer, colorectal cancer, kidney cancer, lung cancer, ovarian cancer, pancreatic cancer or prostate cancer.

The analysing against representative signatures from previous subjects may comprise applying a trained model to the spectroscopic signature.

The trained model may comprise a trained machine learning model, optionally a neural network, a support vector machine (SVM), a random forest (RF) decision tree, an ordered random forest (ORF) model. Alternatively, or additionally, any other suitable model, or model features, may be used for example ORF-PLS, ORF-SVM and/or bagging models, shrinkage discriminant analysis or distance weighed discrimination (DWD) linear models.

The trained model may comprise or functions as a classifier by applying the, or a, probability threshold to a probability value output by the trained model.

The method may further comprise selecting and/or varying the probability threshold thereby selecting and/or varying the specificity and/or sensitivity of the spectroscopic signature analysis.

The method may further comprise selecting the probability threshold based on a receiver operating characteristic (ROC) curve for the trained model, and/or selecting the trained model from a set of trained models based on ROCs for the set of trained models, for example thereby to obtain a desired specificity and/or sensitivity.

One method of detecting presence of cancer, and optionally the type of cancer, in a subject using the method as described in the present disclosure is through the analysis of molecular vibrational modes obtained using ATR-IR analysis of the subject's blood sample or similar methods.

In one embodiment, the method of detecting presence of cancer in a subject, and optionally detection of specific type of cancer, comprises analysis of molecular vibrational modes selected from: N—H (in-plane) bend/deformation, C—N stretch, C—H stretch/deformation, CH₂ stretch, C—O stretch, C—C stretch, C—OH deformation, CH₂ wagging, C═O stretch, asymmetric PO₂⁻ stretch and/or symmetric PO₂⁻ stretch.

In one embodiment, the method of detecting whether or not the subject has cancer is based on the analysis of one or more vibration modes of the spectroscopic signature selected from: C—O stretch, C—C stretch, C—H deformation, N—H bend, C—N stretch and/or C═O stretch. In another embodiment, the method of detecting whether or not the subject has cancer is based on the analysis of vibration modes of the spectroscopic signature comprising C—O stretch, C—C stretch, C—H deformation, N—H bend, C—N stretch and C═O stretch.

In one embodiment, the method of detecting brain cancer in a subject is based on the analysis of one or more vibration modes of the spectroscopic signature selected from: N—H bend, C—N stretch, C—H stretch, and/or CH₂ stretch. In another embodiment, the method of detecting brain cancer in a subject is based on the analysis of vibration modes of the spectroscopic signature comprising N—H bend, C—N stretch, C—H stretch and CH₂ stretch.

In one embodiment, the method of detecting breast cancer in a subject is based on the analysis of one or more vibrational modes of the spectroscopic signature selected from: C—O stretch, C—C stretch, C—OH deformation, N—H in-plane bend, C—N stretch, asymmetric PO₂⁻ stretch and/or CH₂ wagging. In another embodiment, the method of detecting breast cancer in a subject is based on the analysis of vibrational modes of the spectroscopic signature comprising C—O stretch, C—C stretch, C—OH deformation, N—H in-plane bend, C—N stretch, asymmetric PO₂⁻ stretch and CH₂ wagging.

In one embodiment, the method of detecting colorectal and/or kidney cancer in a subject is based on the analysis of one or more vibrational modes of the spectroscopic signature selected from: C═O stretch, C—N stretch, N—H bend, C—O stretch, C—C stretch and/or C—H deformation. In another embodiment, the method of detecting colorectal and/or kidney cancer in a subject is based on the analysis of vibrational modes of the spectroscopic signature comprising C═O stretch, C—N stretch, N—H bend, C—O stretch, C—C stretch and C—H deformation.

In one embodiment, the method of detecting lung cancer in a subject is based on the analysis of one or more vibrational modes of the spectroscopic signature selected from: symmetric/asymmetric PO₂⁻ stretch, C—O stretch, N—H in-plane bend and/or C—N stretch. In another embodiment, the method of detecting lung cancer in a subject is based on the analysis of vibrational modes of the spectroscopic signature comprising symmetric/asymmetric PO₂⁻ stretch, C—O stretch, N—H in-plane bend and C—N stretch.

In one embodiment, the method of detecting ovarian cancer in a subject is based on the analysis of one or more vibrational modes of the spectroscopic signature selected from: N—H bend/deformation, C—N stretch, C—O stretch, C—H deformation and/or C—C stretch. In another embodiment, the method of detecting ovarian cancer in a subject is based on the analysis of vibrational modes of the spectroscopic signature comprising N—H bend/deformation, C—N stretch, C—O stretch, C—H deformation and C—C stretch.

In one embodiment, the method of detecting pancreatic cancer in a subject is based on the analysis of one or more vibrational modes of the spectroscopic signature selected from: N—H bend/in-plane bend, C—N stretch, symmetric Po stretch and/or C—O stretch. In another embodiment, the method of detecting pancreatic cancer in a subject is based on the analysis of vibrational modes of the spectroscopic signature comprising N—H bend/in-plane bend, C—N stretch, symmetric Po stretch and C—O stretch.

In one embodiment, the method of detecting prostate cancer in a subject is based on the analysis of one or more vibrational modes of the spectroscopic signature selected from: C—O stretch, C—H deformation, N—H bend/deformation, C—N stretch and/or C═O stretch. In another embodiment, the method of detecting prostate cancer in a subject is based on the analysis of vibrational modes of the spectroscopic signature comprising C—O stretch, C—H deformation, N—H bend/deformation, C—N stretch and C═O stretch.

An alternative spectroscopic feature that may be used to detect presence of cancer in a subject, and optionally the type of cancer in subjects with cancer, is through identification of peaks at distinct wavenumber regions in the spectroscopic signature of the sample from the subject.

In one embodiment, the method for detecting whether or not the subject has cancer and if the subject has cancer, the type of cancer, is based on the identification of peaks in the spectroscopic signature of the sample at one or more wavenumber regions within 400-4000 cm⁻¹ or a portion or portions thereof. In an alternative embodiment, the method for detecting whether or not the subject has cancer and if the subject has cancer, the type of cancer, is based on the identification of peaks in the spectroscopic signature of the sample at one or more wavenumber regions within 1000-3700 cm⁻¹ or a portion or portions thereof.

In one embodiment, the method of detecting whether or not the subject has cancer is based on identification of peaks at one or more wavenumbers: 1163 cm⁻¹, 1578 cm⁻¹ and/or 1682 cm⁻¹. In another embodiment, the method of detecting whether or not the subject has cancer is based on identification of peaks at 1163 cm⁻¹, 1578 cm⁻¹ and 1682 cm⁻¹.

In one embodiment, the method of detecting whether or not the subject has brain cancer is based on identification of peaks at one or more wavenumbers: 1595 cm⁻¹, 2930 cm⁻¹ and/or 1525 cm⁻¹. In another embodiment, the method of detecting whether or not the subject has brain cancer is based on identification of peaks at 1595 cm⁻¹, 2930 cm⁻¹ and 1525 cm⁻¹.

In one embodiment, the method of detecting whether or not the subject has breast cancer is based on identification of peaks at one or more wavenumbers: 1025 cm⁻¹, 1260 cm⁻¹ and/or 1337 cm⁻¹. In another embodiment, the method of detecting whether or not the subject has breast cancer is based on identification of peaks at 1025 cm⁻¹, 1260 cm⁻¹ and 1337 cm⁻¹.

In one embodiment, the method of detecting whether or not the subject has colorectal cancer is based on identification of peaks at one or more wavenumbers: 1682 cm⁻¹, 1575 cm⁻¹ and/or 1165 cm⁻¹. In another embodiment, the method of detecting whether or not the subject has colorectal cancer is based on identification of peaks at 1682 cm⁻¹, 1575 cm⁻¹ and 1165 cm⁻¹. In one embodiment, the method of detecting whether or not the subject has kidney cancer is based on identification of peaks at one or more wavenumbers: 1682 cm⁻¹, 1590 cm⁻¹ and/or 1164 cm⁻¹. In another embodiment, the method of detecting whether or not the subject has kidney cancer is based on identification of peaks at 1682 cm⁻¹, 1590 cm⁻¹ and 1164 cm⁻¹.

In one embodiment, the method of detecting whether or not the subject has lung cancer is based on identification of peaks at one or more wavenumbers: 1074 cm⁻¹, 1190 cm⁻¹ and/or 1288 cm⁻¹. In another embodiment, the method of detecting whether or not the subject has lung cancer is based on identification of peaks at 1074 cm⁻¹, 1190 cm⁻¹ and 1288 cm⁻¹.

In one embodiment, the method of detecting whether or not the subject has ovarian cancer is based on identification of peaks at one or more wavenumbers: 1569 cm⁻¹, 1412 cm⁻¹ and/or 1135 cm⁻¹. In another embodiment, the method of detecting whether or not the subject has ovarian cancer is based on identification of peaks at 1569 cm⁻¹, 1412 cm⁻¹ and 1135 cm⁻¹.

In one embodiment, the method of detecting whether or not the subject has pancreatic cancer is based on identification of peaks at one or more wavenumbers: 1577 cm⁻¹, 1074 cm⁻¹ and/or 1289 cm⁻¹. In another embodiment, the method of detecting whether or not the subject has pancreatic cancer is based on identification of peaks at 1577 cm⁻¹, 1074 cm⁻¹ and 1289 cm⁻¹.

In one embodiment, the method of detecting whether or not the subject has prostate cancer is based on identification of peaks at one or more wavenumbers: 1356 cm⁻¹, 1499 cm⁻¹ and/or 1642 cm⁻¹. In another embodiment, the method of detecting whether or not the subject has prostate cancer is based on identification of peaks at 1356 cm⁻¹, 1499 cm⁻¹ and 1642 cm⁻¹.

In one embodiment, any of the abovementioned wavenumber regions may vary by ±100 cm⁻¹, ±50 cm⁻¹, ±40 cm⁻¹, ±30 cm⁻¹, ±20 cm⁻¹ and/or ±10 cm⁻¹.

The spectroscopic signature may be correlated with a favourable or unfavourable diagnosis and/or prognosis based on a predictive model developed by “training” (e.g. via pattern recognition and/or machine learning algorithms) a database of pre-correlated analyses. In order to train a model, we provide known cancer and known non-cancer samples from the appropriate patient population in order to identify the signature that can enable discrimination. Correlating the analytical results with a favourable or unfavourable diagnosis and/or prognosis may be performed manually (e.g. by a clinician or other suitable analyst) or automatically (e.g. by computational means). Correlations may be established qualitatively (e.g. via a comparison of graphical traces or signatures) or quantitatively (e.g. by reference to predetermined threshold values or statistical limits). Correlating the analytical results may be performed using a predictive model, optionally as defined herein, which may have been developed by “training” a database of pre-correlated assays and/or analyses.

In one embodiment, a computer program product may be provided comprising computer-readable instructions that are executable to perform the method as described herein. A trained machine learning model may be configured to receive an input comprising a spectroscopic signature of an IR spectroscopic analysis comprising wavelengths between 400-4000 cm⁻¹ performed on a blood sample from the subject and to provide an output representative of whether or not the subject has a cancer. In one embodiment, the method of training a machine learning model may comprise receiving a plurality of data sets representing spectroscopic signatures for a plurality of subjects obtained from IR spectroscopic analysis comprising wavelengths between 400-4000 cm⁻¹ on blood samples from the subjects; receiving cancer data indicating for at least some of the subjects whether the subject has cancer; and training the model to determine a probability of whether a patient has cancer based on a spectroscopic signature for the patient, wherein some of the subjects have cancer and the subjects with cancer comprise subjects with different types of cancer and different stages of cancer. The training of the model may also comprise tuning at least one parameter of the model to optimise the area under the ROC curve and/or to provide a desired sensitivity and/or specificity for a determination as to whether a patient has cancer.

The multi-cancer detection method of the present disclosure provides a method of analysing macromolecules in a minute volume of patient serum through IR spectroscopic analysis and machine learning algorithms. As the analysis only requires a small volume of blood, it may be feasible to integrate this rapid liquid biopsy spectroscopic test with other existing blood-based tests without disrupting clinical practice, such as taking a small aliquot of blood from a routine blood test. In one embodiment, the small aliquot taken from the blood sample for IR analysis may typically be less than 1 mL. In alternative embodiments, the small aliquot taken from the blood sample for IR analysis may be less than 100 μL, 80 μL, 60 μL, 40 μL or 10 μL. In this manner, the bulk (typically over 80%, 90%, 95%, or 99%) of the original sample is still available for subsequent analysis.

The methods of the present disclosure may therefore permit effective triage of patients, by expediting further assessment for patients more at risk while excluding a cancer diagnosis in others. In addition, it may identify early-stage cancer in asymptomatic patients or patients with non-specific symptoms if offered in conjunction with other blood based diagnostic assays or routine blood tests. Moreover, the methods of the present disclosure are effective at stratifying subjects with cancer from subjects without cancer, irrespective of the stage of cancer, or the type of cancer. Optionally, the methods may further be effective irrespective of the age and/or the sex of the subjects. In one embodiment, the liquid biopsy based spectroscopic analysis can differentiate subjects with cancer from subjects without cancer independent of the age of the subjects. In another embodiment, the liquid biopsy based spectroscopic analysis can differentiate subjects with cancer from subjects without cancer independent of the sex of the subjects.

In one embodiment, the methods of the present disclosure may be conducted as a standalone test or optionally as an additive test in combination with other blood-based tests.

In one embodiment, the methods as disclosed herein may conducted in combination with one or more other blood-based tests using a single blood draw, wherein a first aliquot is used to conduct the IR spectroscopic analysis as described herein and a second aliquot comprising the remainder of the blood sample, or a portion thereof, is used to conduct one or more other blood-based tests. In one embodiment, the remainder of the blood draw may be used to conduct standard routine blood tests or other liquid biopsy based diagnostic tests. In an alternative embodiment, the further analysis may comprise conducting a follow-up test, such as liquid biopsy sequencing assays (e.g., NGS-based ctDNA tests, methylation tests, biomarker tests). The single blood draw may be aliquoted into separate containers or receptacles, or the single blood draw may be provided into a single container or receptacle.

In a further aspect, there is provided a computer program product comprising computer readable instructions that are executable to perform a method as claimed or described herein.

In another aspect, which may be provided independently, there is provided a trained model configured to receive an input comprising a spectroscopic signature of an ATR-IR spectroscopic analysis comprising wavelengths between 400-4000 cm⁻¹ performed on a blood sample from the subject and to provide an output representative of whether or not the subject has a cancer, irrespective of the stage or type of cancer.

In another aspect, which may be provided independently, there is provided a method of training a model comprising receiving a plurality of data sets representing spectroscopic signatures for a plurality of subjects obtained from ATR-IR spectroscopic analysis comprising wavelengths between 400-4000 cm⁻¹ on blood samples from the subjects; receiving cancer data indicating for at least some of the subjects whether the subject has cancer; and training the model to determine a probability of whether a patient has cancer based on a spectroscopic signature for the patient, wherein some of the subjects have cancer and the subjects with cancer comprise subjects with different types of cancer and different stages of cancer.

The training of the model may comprise tuning at least one parameter of the model to optimise the area under the ROC curve and/or to provide a desired sensitivity and/or specificity for a determination as to whether a patient has cancer.

Features in any one aspect may be provided as features in any one or more other aspects. For example, any of method, computer program product, model or apparatus features may be provided as any one or more other of method, computer program product, model or apparatus features.

### DETAILED DESCRIPTION

The present disclosure will now be further described by way of example and with reference to the Tables and Figures, which show:

Table 1. Patient demographics for the cancer (C) vs non-cancer (NC) classification.

Table 2. Detection rates from the sensitivity-tuned model for each cancer class, split by stage, based upon the models with a lower limit of 45% specificity for the cross validation.

Table 3. Detection rates for the sensitivity and specificity-tuned models split by age and sex.

Table 4. Patient demographics for the organ specific cancer (C) v non-cancer symptomatic (NCS) classifications.

Table 5. Patient demographics for the organ specific classifications for ovarian and prostate cancer against the sex-specific non-cancer symptomatic (NCS) groups.

Table 6. Sensitivity and specificity values for the resampled test sets for each of the organ specific cancer classification. The results here are based upon thresholds chosen where either sensitivity or specificity was 90% for the cross validation. Non-cancer symptomatic (NCS), NCS female-only (NCS-F) and NCS male-only (NCS-M).

Table 7. Detection rates from the organ-specific cancer classification, for the specificity-tuned models split by stage. The results here are based upon a lower limit of 45% for the cross-validation sensitivity.

Table 8. The top 3 wavenumber regions that were found to be the most discriminatory for each of the binary classifications, with their corresponding biological assignments and vibrational modes.

Table 9. The top 5 wavenumber regions which were found to be the most discriminatory for each of the binary classifications, with their corresponding tentative biological assignments and vibrational modes.

Table 10. Summary of brain cancer types split by tumour grade.

Table 11. 95% confidence intervals (CI) for a one-sample Student's t-test, carried out for each selected threshold on the presented receiver operating characteristic curves for every classification.

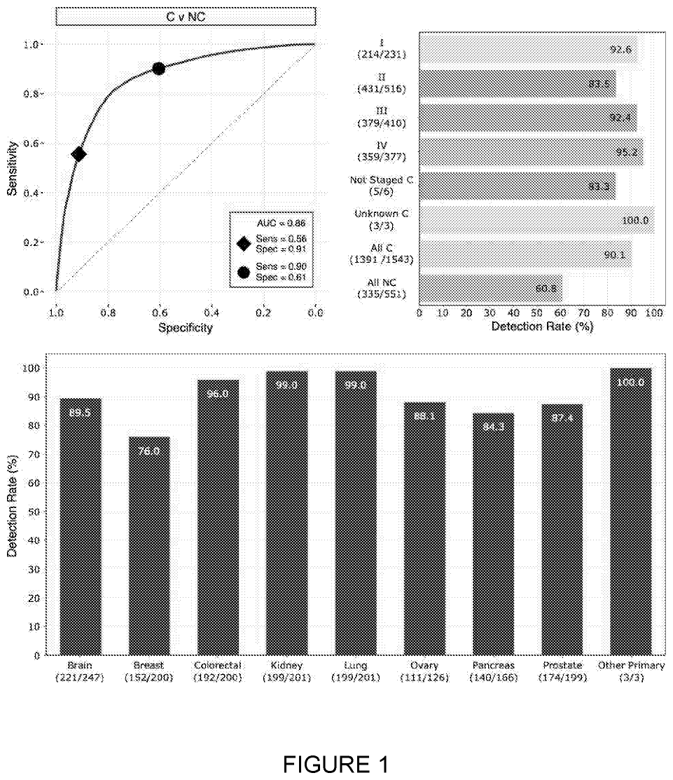

FIG. 1. Results from the cancer (C) versus non-cancer (NC) classification showing (a) the mean receiver operating characteristic curve showing the trade-off between sensitivity (Sens) and specificity (Spec), where the markers represent sensitivity-tuned model (black circle), and specificity-tuned model (black diamond), and AUC denotes the area under the curve; (b) the detection rate of each cancer stage and (c) the detection rate of each cancer type. Both (b) and (c) are shown for the sensitivity-tuned model.

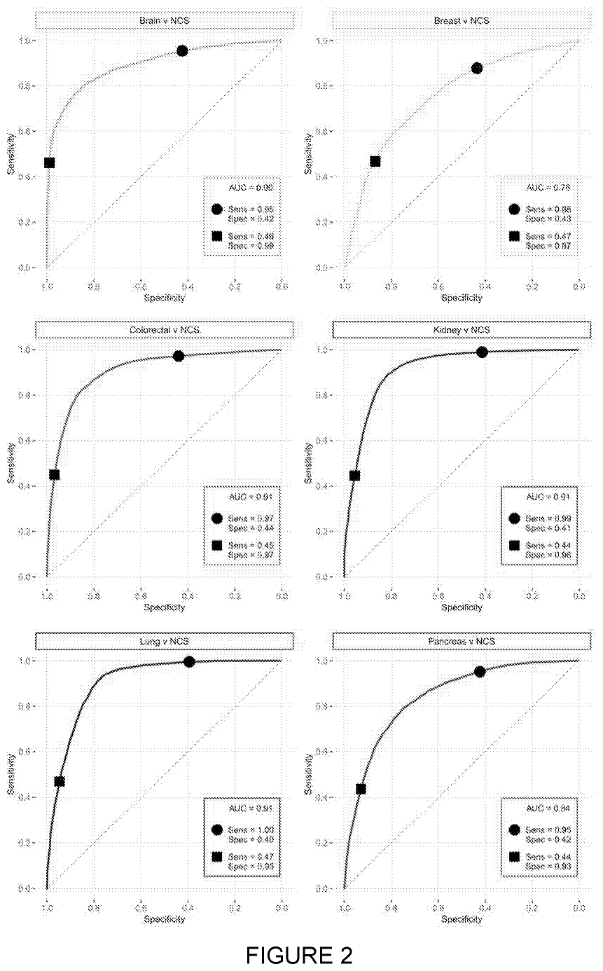

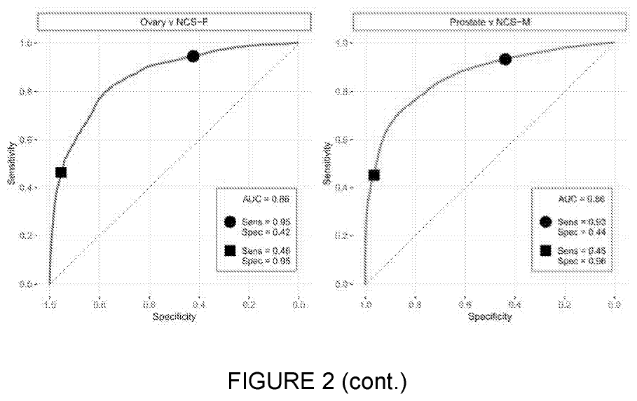

FIG. 2. Mean receiver operating characteristic curves for the organ specific cancer classifications (a) brain, breast, colorectal, kidney, lung, and pancreatic cancer versus non-cancer symptomatic (NCS), and (b) ovary cancer versus NCS female-only (NCS-F) and prostate cancer versus NCS male-only (NCS-M). In all ROC plots the markers represent sensitivity-tuned model (black circle), and specificity-tuned model (black diamond), and AUC denotes the area under the curve.

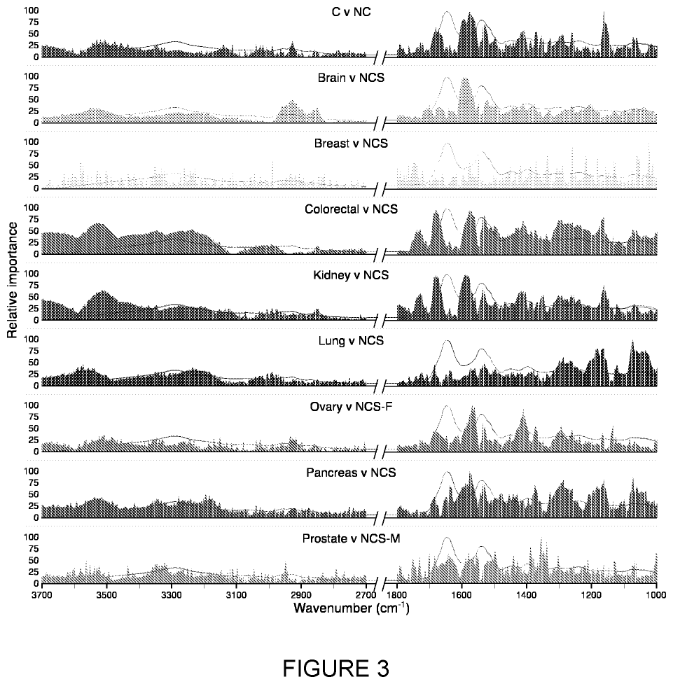

FIG. 3. Feature importance plots highlighting the wavenumber regions that were found to be the most discriminatory for each of the binary classifications.

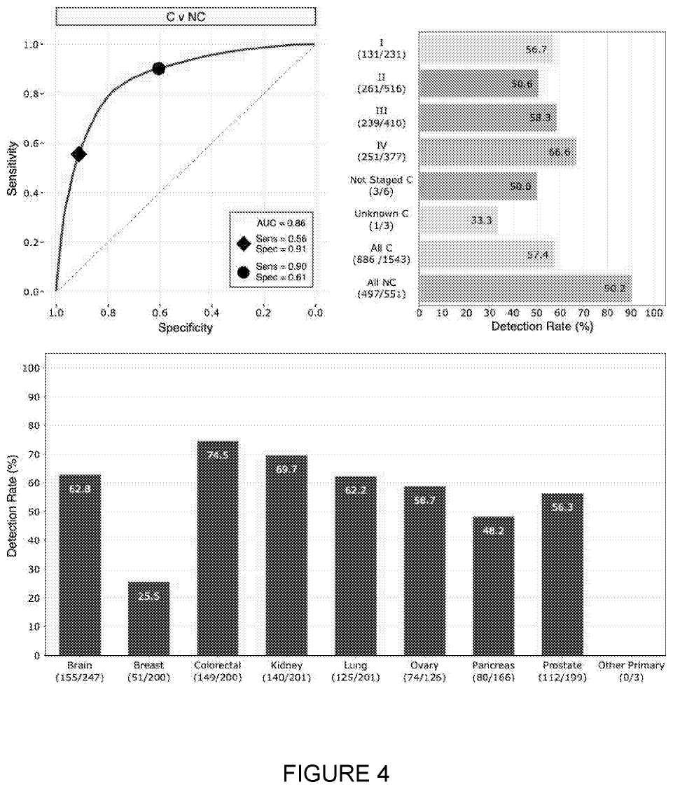

FIG. 4. Results from the cancer (C) versus non-cancer (NC) classification showing (a) the mean receiver operating characteristic curve showing the trade-off between sensitivity (Sens) and specificity (Spec), where the markers represent sensitivity-tuned model (black circle), and specificity-tuned model (black diamond), and AUC denotes the area under the curve; (b) the detection rate of each cancer stage and (c) the detection rate of each cancer type. Both (b) and (c) are shown for the specificity-tuned model.

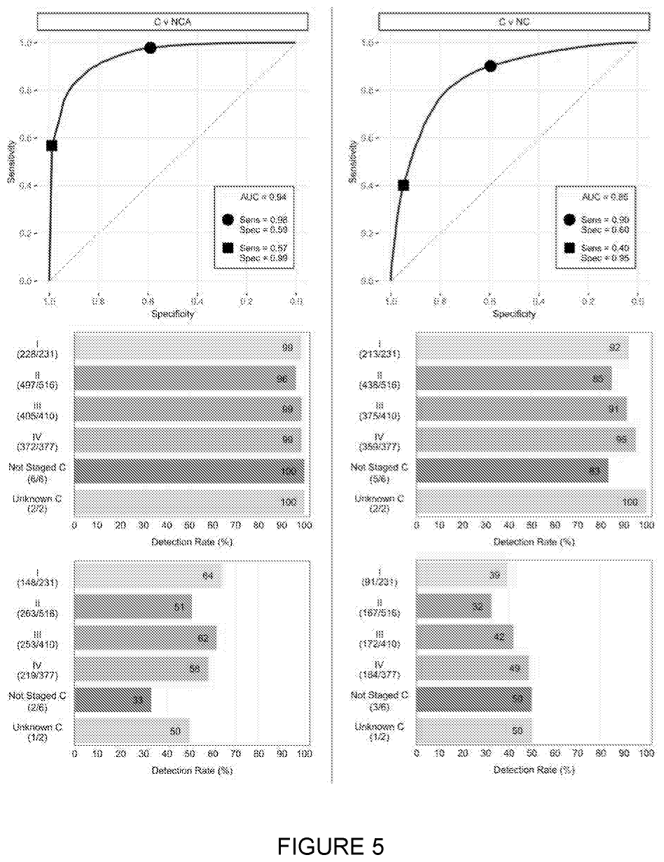

FIG. 5. Results from the cancer (C) versus asymptomatic non-cancer (NCA) classification (a, c, e) and the C versus all non-cancer (NC) classification (b, d, f). The mean receiver operating characteristic curve for a) C v NCA and b) C v NC showing the trade-off between sensitivity (Sens) and specificity (Spec), where the markers represent sensitivity-tuned model (●), and specificity-tuned model (▪), and AUC denotes the area under the curve. The detection rates for the sensitivity-tuned models (c, d) and the specificity-tuned models (e, f) are illustrated for the respective classifications, split by cancer stage

### METHODS

### Patient Sample Cohort Selection

All samples sourced from patients eligible for inclusion in this study were purchased and/or collected from biobanks, commercial sources and active clinical research studies (Lothian REC 15/ES/0094; IRAS 238735). All patients gave written consent for inclusion in the study. The cancer samples were collected from patients with a confirmed cancer diagnosis before surgical resection and advanced therapies. The non-cancer group was comprised of healthy volunteers and patients with suspicious symptomatology, including patients with benign non-cancerous conditions and no evidence of neoplasia—the non-cancer patient serum was sourced during clinical assessment.

Blood samples were obtained with venepuncture using serum collection tubes and anonymised. Serum was extracted via centrifugation and stored in a −80° C. freezer. Clinical and demographic data were obtained respecting participants' confidentiality.

### Patient Sample Analysis

Patient serum samples were analyzed using the Dxcover® Cancer Liquid Biopsy (Dxcover® Ltd., Glasgow, UK) spectroscopic test. Previously conducted clinical studies have employed this test, to which we direct the reader for further information (10). In this study, the serum samples sourced were stored at −80° C. until the date of analysis; samples were allowed to thaw for up to 30 minutes at room temperature (18-25° C.) and inverted three times to ensure sufficient mixture and thawing before use. Each patient sample was prepared for analysis by pipetting 3 μL of serum onto each of the three sample wells of the Dxcover® sample slide (Dxcover® Ltd., Glasgow, UK). Prepared slides were placed in a drying unit incubator (Thermo Scientific^{T}M Heratherm™, Waltham, Massachusetts, US) at 35° C. for 1 hour, to control the dehydration process of the serum droplets (11). Each dried sample slide was then inserted into the Dxcover® autosampler (Dxcover® Ltd., Glasgow, UK) to be prepared for spectral collection. In this study, a PerkinElmer@Spectrum Two^{T}M FTIR spectrometer (PerkinElmer@Inc., Waltham, Massachusetts, USA) was used to generate the spectral data (16 co-added scans at 4 cm⁻¹ resolution with 1 cm⁻¹ data spacing). A total of three spectra were collected for each sample well, resulting in nine replicates per patient, then submitted to the diagnostic algorithm to generate the disease prediction. Patient samples were reported as cancer positive or negative according to the diagnostic algorithm results.

### Algorithm Training

Machine learning models were developed to identify the cancerous signature from a known patient cohort then predict the presence of cancer in an unknown population. A nested cross-validation strategy was used to develop the model, in which the inner cross-validation was used to tune the model hyper-parameters, and the outer cross-validation provided a robust test of model performance. In this approach, patients were randomly split into training and test sets, with a 70:30 split. Model hyper-parameters were tuned to optimise the area under the receiver operator characteristic curve and to give a desired sensitivity or specificity, as estimated from 5-fold cross-validation on the training set (70%). The trained model was used to make predictions for the spectra in the test set (30%). Since each patient sample was analysed nine times, the final diagnosis was taken as the consensus prediction (maximum vote) from all nine spectra. To obtain a robust estimate of the performance of the classifier, the model building process was repeated 51 times using different training and test set splits, and the mean and standard deviation of the resulting classification metrics were recorded.

Any suitable machine learning model, for example any suitable deep learning model, can be used according to embodiments, and can be trained according to any suitable training techniques and using any suitable training data sets. The model may comprise a neural network, for example a convolutional neural network or a recurrent neural network. In some embodiments, the convolutional neural network may comprise one or more of: at least one 1-dimensional convolutional layer, at least one down sampling layer and/or at least one batch normalization layer. The recurrent neural network may comprise one or more long-short term memory (LSTM) layers. Other exemplary models which may be used include algorithms such as partial least squares (PLS) models, logistic regression models, state vector machine (SVM), or LDA models, random forest (RF) decision trees, ordered random forest (ORF) models, ORF-PLS, ORF-SVM, bagging models, shrinkage discriminant analysis and/or distance weighed discrimination (DWD) linear models. The machine learning models, for example deep learning models, may be single models or ensembles of multiple models. Ensemble models may, for example, be built on different samples of the data using one or more of bootstrap aggregating (“bagging”) or random sampling. In some embodiments, two or more machine learning algorithms are applied sequentially where e.g. a cancer prediction from the first algorithm is reclassified by the second algorithm. In this case, any suitable machine learning algorithm could be used for the first or second algorithm.

The trained model may comprise or functions as a classifier by applying the or a probability threshold to a probability value output by the trained model. In some embodiments, the probability threshold is selected and/or varied thereby selecting and/or varying the specificity and/or sensitivity of the spectroscopic signature analysis.

The method may further comprise selecting the probability threshold based on a receiver operating characteristic (ROC) curve for the trained model, and/or selecting the trained model from a set of trained models based on ROCs for the set of trained models, for example thereby to obtain a desired specificity and/or sensitivity.

### Metadata Analysis

Breakdowns by patient metadata, such as those presented in FIG. 1, were performed as post-hoc analyses using the test set predictions from all 51 train-test splits. The test set is randomly sampled from the full dataset, so a given patient would be expected to be present in around 15 test sets out of the 51 total.

For each patient, the predictions from all test sets in which that patient is present were collected, and a detection rate was calculated as the ratio of correct predictions to total number of predictions. The detection rates are then averaged over all patients of each category of metadata (e.g., disease stage or actual disease as in FIG. 1). It is important to note that the resulting values are not directly comparable to the sensitivity values that appear on the ROC curves, which are computed as the mean over all test sets, rather than over a subset.

### EXAMPLES

### Overall Cancer Classification

The full cohort consists of 2092 patients, of which 1542 had a confirmed cancer diagnosis (Table 1). The cancer set is comprised of patients with either brain, breast, colorectal, kidney, lung, ovarian, pancreatic or prostate cancer. 91 of the non-cancer group were healthy volunteers, and the remaining 459 were patients with generic presenting symptoms, such as headache and stroke, as well as other benign conditions, e.g. non-malignant cysts and polyps. A respectable balance of male and female participants has been included, with a widespread age range.

Initially, the algorithm was tuned to selected thresholds that resulted in a 90% sensitivity or specificity for the cross-validation (CV) set. The mean receiver operating characteristic (ROC) curve computed for patient predictions over all resampled test sets, reports an area under the curve (AUC) value of 0.86, which suggests excellent detection capability between cancer and non-cancer (NC) (12). The two points marked on the ROC curve in FIG. 1, represent the sensitivity-tuned model (black circle) which achieves 90% sensitivity and 61% specificity, and the specificity-tuned model (black diamond) which achieves 56% sensitivity and 91% specificity, respectively, for the resampled test sets. We can stratify the results from this classifier to visualize the number of correct predictions that were made. The bar graphs in FIG. 1 represent the detection rate for the sensitivity-tuned model when split by (b) stage and (c) cancer type. The equivalent plots for the specificity-tuned model are shown in FIG. 4.

The most important feature of a multi-cancer test is that it must be capable of detecting early-stage cancer. FIG. 1 also illustrates that when tuned for 90% CV sensitivity, the Dxcover® Cancer Liquid Biopsy detected 93% (214/231) of stage I and 84% (431/516) of stage II cancers. Similarly, the detection rate was extremely high for the later stage cancers—92% (379/410) and 95% (359/377) for stage III and IV, respectively. For all cancers combined, 90.1% (1391/1543) were predicted correctly. With regards to cancer type, the graph in FIG. 1 demonstrates the detection capability of the Dxcover® Cancer Liquid Biopsy to detect a wide variety of cancers—brain (89%), breast (76%), colorectal (96%), kidney (99%), lung (99%), ovary (88%), pancreas (84%) and prostate (87%) cancer.

We next tuned for maximum sensitivity or specificity, whilst the other statistic was fixed above a minimum value of 45% for the CV result. This resulted in a 94% sensitivity where specificity was 47%, and a 94% specificity with 48% sensitivity. By increasing the sensitivity to 94%, the test would detect more cancers, however this would also increase the number of false positives, with the specificity dropping from 61% to 47%. Additionally, patient metadata factors were explored to assess any impact on the predictions of the multi-cancer liquid biopsy. Patient age did not significantly affect either the sensitivity-tuned or specificity-tuned models (Table 3). Likewise, the detection rates for both models when split by sex did not indicate any concerns as a potential confounding factor.

FIG. 5 shows results from an alternative cancer (C) versus asymptomatic non-cancer (NCA) classification (a, c, e) and the C versus all non-cancer (NC) classification (b, d, f).

This C v NCA algorithm was tuned to selected thresholds that resulted in a 98% sensitivity or specificity for the CV set. The C v NCA ROC analysis reported an AUC value of 0.94, which suggests excellent detection capability (FIG. 5a). This results in a 98% sensitivity (59% specificity) or a specificity of 99% (57% sensitivity). Liquid biopsies that target the asymptomatic screening populations are currently aiming for high specificities to minimize false positives. However, in a symptomatic population, a triage test may be more appropriate, therefore the selected probability thresholds for the overall C v NC classifier—which incorporates both asymptomatic and symptomatic non-cancer patients—were chosen for 90% sensitivity and 95% specificity for the CV set. For the C v NC dataset (FIG. 5b), the sensitivity-tuned model achieved 90% sensitivity and 60% specificity, and when tailored for greater specificity (95%) the sensitivity was 40%. The ROC curve generated an AUC of 0.85.

The bar graphs in FIG. 5 represent the detection rate when split by stage: (c), (d) sensitivity-tuned and (e), (f) specificity-tuned results for the C v NCA and C v NC classifiers, respectively. When exploring C v NCA, the sensitivity-tuned model successfully predicted 98% of all cancers correctly. The detection rates were consistent across all stages: stage I, 99%; II, 96%; III, 99%; IV, 99%. On the other hand, the high specificity (99%) model was still capable of detecting 64% of stage I cancers and identified 51% of stage II. Therefore, 55% of stage I-II cancers were predicted correctly, highlighting the great potential for the Dxcover® Cancer Liquid Biopsy in the detection of early-stage cancers. For the overall C v NC classification, FIG. 5d illustrates that when tuned for higher sensitivity, 92% (213/231) of stage I and 85% (438/516) of stage II cancers were detected. Similarly, the detection rate was extremely high for the late-stage cancers—91% (375/410) and 95% (359/377) for stage III and IV, respectively. For the model with 95% specificity, the detection rates are fairly consistent across stages: I 39%; II 32%; III 42%; IV 49% (FIG. 5f).

### Organ Specific Cancer

The ability of the liquid biopsy to differentiate organ specific cancers from the non-cancer patients that had presenting symptoms (NCS) with binary classifications was next examined. A symptomatic cohort is more applicable to real-world triage testing, thus is a more relevant comparator than healthy participants. Several conditions are encompassed in the NCS set, such as stroke, inflammation, and seizures, as well as benign polyps and cysts. The brain, breast, colorectal, kidney, lung, and pancreatic cancer groups were tested against the full NCS dataset which includes a balance of both male and female patients (Table 4). The ovary cancer set was compared against the female participants in the NCS group (NCS-F), and the prostate cancer group was examined against the male-only NCS patients (NCS-M), as described in Table 5.

Not all cancers require the same approach. The optimal test sensitivity and specificity for individual cancers may be influenced by the availability, costs and risks of subsequent diagnostic investigations, or whether the test would impact on a current screening programme (13). Similar to the overall cancer classification, probability thresholds were selected which produced 90% sensitivity or specificity for the CV of each organ specific classifier, and the sensitivity and specificity value for the resampled test sets have been reported (Table 6). Notably, the classifiers for colorectal (91% sensitivity/72% specificity), lung (91% sensitivity/77% specificity) and kidney (92% sensitivity/75% specificity) cancer show real promise for organ specific applications with well-balanced sensitivities and specificities. We then tuned for greater sensitivity or specificity by employing a lower limit of 45% for the CV result, which was selected based on current commercially available triage-based liquid biopsies. The mean ROC curves of the test sets for each of the organ specific classifiers are illustrated in FIG. 2, with the two points on the curve corresponding to the sensitivity-tuned (black circle) and specificity-tuned (black diamond) models, along with their corresponding AUC values. The brain, colorectal, kidney, and lung cancer versus NCS classifications reported very promising results, with AUCs of 0.90 and above. The pancreatic cancer versus NCS model achieved an AUC of 0.84. The breast cancer versus NCS model achieved an AUC of 0.76; however, this still yields a sensitivity of 88% when specificity is 43%, and a specificity of 87% when sensitivity is 47%. The ovary and prostate cancer models both performed very well and both reported an AUC of 0.86. For each of the organ specific classifications, the predictions were examined by cancer stage. The detection rates were calculated for both the sensitivity-tuned (Table 2) and specificity-tuned (Table 7) models. For every classification in this study, 95% confidence intervals (CI) were calculated for each selected threshold on each ROC curve, as shown in Table 11.

The sensitivity-tuned results are described in Table 2. The brain cancer detection rate was 100% (8/8) for grade I, 85% (23/27) for grade II, 86% (12/14) for grade III and 99% (191/192) for grade IV, which had the overwhelming majority for the brain cancer set. The breast cancer group had many more early-stage samples, and the detection rate was 96% (24/25) for stage I, 87% (79/91) for stage II, 89% (67/75) for stage III and 100% (9/9) for stage IV. The classifiers for colorectal, kidney and lung cancer all reported extremely high detection rates, between 98-100% for stage I and II. Despite being the smallest subset in this study, the ovarian cancer predictions were still highly promising: stage I 97% (30/31); II 86% (12/14); III 92% (47/51); IV 100% (29/29). Likewise, due to difficulty sourcing stage I pancreatic cancer samples there were only 8 included in the dataset, yet the sensitivity-tuned model was still capable of successfully predicting 7 of the stage I tumors (88%). Additionally, the detection rates for the other pancreatic cancer stages were 94% (II, 61/65) 99% (III, 71/72) and 95% (IV, 19/20). Lastly, the prostate cancer results further highlighted the potential of earlier detection: stage I 100% (4/4); II 93% (149/160); III 97% (30/31); IV 75% (3/4).

For the models that were tailored for a greater specificity (Table 7), more of the cancer samples were not detected. The brain cancer detection rates were low for grade I-III yet reported 52% (100/192) for grade IV (at 99% specificity). The breast cancer group had detection rates of 32% (8/25) for stage I, 47% (43/91) for stage II, 59% (44/75) for stage III, and 78% (7/9) for stage IV. The colorectal classifier reported the highest stage I detection rate for the specificity-tuned models with 18 out of 36 being predicted correctly (50%). Furthermore, the colorectal stage II, III and IV detection rates were 36% (25/70), 51% (34/67) and 44% (12/27), respectively. Kidney cancer reported the highest detection rate for stage II (66%, 19/29), and the remaining stages were: stage I 46% (40/87); III 38% (13/34); IV 29% (15/51). The detection rates for lung cancer were 35% (11/31) for stage I, 40% (24/60) for stage II, 60% (39/65) for stage III and 44% (20/45) for stage IV. The detection rates were similar for ovarian cancer—stage I 39% (12/31), II 36% (5/14), III 53% (27/51), IV 45% (13/29)—and pancreatic cancer—stage I 38% (3/8), II 37% (24/65), III 44% (32/72), IV 55% (11/20). Finally, the prostate cancer performance was respectable for stage II (43%, 68/160) and III (58%, 18/31), but low for stage I and IV which is likely to be attributed to the very small number of samples in those groups (n=4).

Advantageously, detections rates were found to be relatively stable across all four stages of disease. Other detection techniques involving liquid biopsies tend to exhibit increasing sensitivity moving from Stage I to Stage IV. This is based on the accepted belief in the field that, as cancer increases, the amount of diagnostic information (e.g. PSA or any genetic markers) increases in concentration and therefore other liquid biopsies should observe and increase in detection accuracy across stages. In contrast, the present dataset of representative signatures includes several stages, preferably all stages I-IV, and the trained model is capable of distinguishing between cancer and non-cancer regardless of cancer stage.

### Feature Importance

When biological samples are irradiated with IR light, the absorbance of this light causes molecular excitation and enables transitions between vibrational states, resulting in an IR spectrum. Biomolecules within blood serum vibrate in distinct planes, such as stretching and bending of the bonds between chemical functional groups, and these vibrations are characteristic of these biomolecules. The spectral regions, or specific wavenumbers that contribute to a classification can be assessed by feature importance analysis. The wavenumber regions that were found to be most discriminatory can be visualized in importance plots (FIG. 3) and the top 3 regions of importance for each of the binary classifications are shown in Table 8 and Table 9. FIG. 3 illustrates the wavenumber regions that were found to be the most discriminatory. The importance values were normalized to a maximum of 100 to make them more comparable. The variable importance measure here is based on weighted sums of the absolute regression coefficients. These are weighted according to the sums of squares across the number of partial least squares (PLS) components chosen by the discriminant model. For C versus NC, the wavenumber regions deemed to be of highest importance were the Amide I (˜1682 cm⁻¹) and Amide II (˜1578 cm⁻¹) bands. These are the two largest peaks in the serum spectrum and contain information from overlapping bands associated with protein secondary structures, such as α-helices and β-sheets, thus variations in these bands are often indicative of disease states (9). They are associated with NH bending vibrations, and CN and CO stretching vibrations in protein molecules. Additionally, for the C versus NC model, CO and CC stretching and CH deformation vibrations related to proteins and carbohydrates arising at the lower wavenumber end of the spectrum (˜1163 cm⁻¹) was found to be of high importance. The brain cancer versus NCS classification reported similar importance within the Amide II region, but also the lipid bands in the high wavenumber region were shown to be significant in this model, which arise at ˜2930 cm⁻¹ and account for CH and CH₂ stretching vibrations. The importance values for the breast cancer model were far more widespread and had many narrow spikes across the full spectrum in comparison to other cancers. The top 3 regions of importance were ˜1025 cm⁻¹, which is associated with glycogen and carbohydrates (CO and CC stretch, COH deformation), the Amide III of proteins (˜1260 cm⁻¹) NH in-plane bend, CN stretching vibrations, and ˜1337 cm⁻¹ accounting for CH₂ wagging and CO stretching vibrations in phospholipids and collagen. The importance trace for the colorectal and kidney cancer models are very similar, the most important regions in both of these classifications were the Amide I and II bands, and other proteinaceous and carbohydrate vibrations around 1165 cm⁻¹. The lung cancer versus NCS feature importance seemed rather unique as most of the important bands appear in the lower end of the spectrum, mainly associated with symmetric (1074 cm⁻¹) and asymmetric (1190 cm⁻¹) PO stretching vibrations, as well as protein-related vibrations in the Amide III region (1288 cm⁻¹). Ovarian and pancreatic cancer importance values were also quite similar, as the Amide II band is of highest importance for both, yet they differ in the other top wavenumber regions. For the ovary versus NCS-F model, ˜1412 cm⁻¹ and ˜1135 cm⁻¹ were also found to be significant, whereas 1074 cm⁻¹ (symmetric PO stretch, C—O stretch associated with nucleic acids and phosphodiesters), and 1289 cm⁻¹ were more important for the pancreas versus NCS classifier. Lastly, the importance values in the prostate versus NCS-M model were mainly associated with protein vibrations (Amide I/II), and the CO stretch with CH and NH deformation within lipids and proteins arising around ˜1356 cm⁻¹

### CONCLUSIONS

The development of a robust multi-cancer detection test would be transformational within the cancer diagnostics field, as earlier detection is vital to inhibit disease progression, improve patient prognosis and reduce mortality rates. In this large-scale spectroscopic study, the inventors present the Dxcover® Cancer Liquid Biopsy, believed to be the world's first infrared spectroscopy-based blood test for the early detection of multiple cancers. For the binary classifiers of individual cancer types against symptomatic control patients, promising AUC values were reported for all cancers: brain (0.90), breast (0.74), colorectal (0.91), kidney (0.91), lung (0.90), ovarian (0.85), pancreatic (0.81) and prostate (0.85). The results presented here show great potential for this spectroscopic method to be utilised for the early detection of multiple cancers. Importantly, the detection rates did not vary significantly according to cancer stage, suggesting the technology is very effective at detecting early-stage tumours, which is a necessity for early cancer detection and patient survival.

TABLE 1

| | | Asymptomatic | | |
|---|---|---|---|---|
| | Cancer (C) | Non-cancer | Non-cancer (NC) | Total* |
| | (n = 1542) | (NCA) | (n = 550) | (n = 2092) |
| Age, years | | | | |
| Mean | 63 | 41 | 54 | 61 |
| Min-max | 20-90 | 20-82 | 20-90 | 20-90 |
| Sex, n (%) | | | | |
| Female | 748 (49) | 50 (55) | 310 (56) | 1058 (51) |
| Male | 794 (51) | 41 (45) | 240 (44) | 1034 (49) |
| Cancer stage, n (%) | | | | |
| I | 231 (15) | — | — | 231 (11) |
| II | 516 (33) | — | — | 516 (25) |
| III | 410 (27) | — | — | 410 (20) |
| IV | 377 (24) | — | — | 377 (18) |
| Not staged** | 6 (1) | 91 (100) | 550 (100) | 556 (26) |
| Unknown*** | 2 (0) | — | — | 2 (0) |

*Total includes C and NC only; NCA patients are comprised within the NC set.
**Does not require staging.
***Cancer stage and/or staging information has not been recorded.

TABLE 2

| | | | | Detection Rate |
|---|---|---|---|---|
| Cancer Type | Stage | Actual | Identified | (%) |
| Brain*** | I | 8 | 8 | 100 |
| | II | 27 | 23 | 85 |
| | III | 14 | 12 | 86 |
| | IV | 192 | 191 | 99 |
| | Not Graded * | 6 | 5 | 83 |
| Breast | I | 25 | 24 | 96 |
| | II | 91 | 79 | 87 |
| | III | 75 | 67 | 89 |
| | IV | 9 | 9 | 100 |
| Colorectal | I | 36 | 36 | 100 |
| | II | 70 | 70 | 100 |
| | III | 67 | 65 | 97 |
| | IV | 27 | 24 | 89 |
| Kidney | I | 87 | 86 | 99 |
| | II | 29 | 29 | 100 |
| | III | 34 | 34 | 100 |
| | IV | 51 | 50 | 98 |
| Lung | I | 31 | 31 | 100 |
| | II | 60 | 59 | 98 |
| | III | 65 | 65 | 100 |
| | IV | 45 | 45 | 100 |
| Ovary | I | 31 | 30 | 97 |
| | II | 14 | 12 | 86 |
| | III | 51 | 47 | 92 |
| | IV | 29 | 29 | 100 |
| Pancreas | I | 8 | 7 | 88 |
| | II | 65 | 61 | 94 |
| | III | 72 | 71 | 99 |
| | IV | 20 | 19 | 95 |
| | Unknown** | 1 | 1 | 100 |
| Prostate | I | 4 | 4 | 100 |
| | II | 160 | 149 | 93 |
| | III | 31 | 30 | 97 |
| | IV | 4 | 3 | 75 |

* Does not require staging.
**Cancer stage and/or staging information has not been recorded.
***Brain cancers split by grade, as described in Table 10

TABLE 3

| | | | | Correctly identified n (%) | | | | | | | |
|---|---|---|---|---|---|---|---|---|---|---|---|
| | | | | | | | | Specificity-tuned | | | |
| | | | | Sensitivity-tuned Model | | | | Model | | | |
| | | No. of Patients | | (90% Sensitivity) | | | | (95% Specificity) | | | |
| | | C | NC | C | | NC | | C | | NC | |
| Sex | Male | 794 | 240 | 732 | (92) | 153 | (64) | 327 | (41) | 229 | (95) |
| | Female | 748 | 310 | 660 | (88) | 184 | (59) | 291 | (40) | 296 | (95) |
| Age | 20 | 15 | 52 | 13 | (87) | 42 | (81) | 6 | (40) | 52 | (100) |
| (deciles) | 30 | 37 | 65 | 28 | (76) | 48 | (74) | 14 | (38) | 65 | (100) |
| | 40 | 140 | 66 | 118 | (84) | 44 | (67) | 47 | (34) | 62 | (94) |
| | 50 | 331 | 84 | 294 | (89) | 49 | (58) | 118 | (36) | 81 | (96) |
| | 60 | 539 | 105 | 490 | (91) | 52 | (50) | 219 | (41) | 98 | (93) |
| | 70 | 373 | 116 | 347 | (93) | 63 | (54) | 170 | (46) | 107 | (92) |
| | 80 | 104 | 54 | 100 | (96) | 34 | (63) | 43 | (41) | 53 | (98) |
| | 90 | 3 | 4 | 2 | (67) | 2 | (50) | 1 | (33) | 3 | (75) |

TABLE 4

| | Brain*** | | Breast | | Colorectal | | Kidney | | Lung | | Pancreas | | NCS | |
|---|---|---|---|---|---|---|---|---|---|---|---|---|---|---|
| | (n = 247) | | (n = 200) | | (n = 200) | | (n = 201) | | (n = 201) | | (n = 166) | | (n = 459) | |
| Age, years | | | | | | | | | | | | | | |
| Mean | 60 | | 62 | | 66 | | 62 | | 66 | | 65 | | 57 | |
| Min-max | 22-84 | | 28-90 | | 29-85 | | 25-87 | | 20-86 | | 39-87 | | 20-90 | |
| Sex, n (%) | | | | | | | | | | | | | | |
| Female | 123 | (50) | 196 | (98) | 64 | (32) | 75 | (37) | 95 | (47) | 69 | (42) | 260 | (57) |
| Male | 124 | (50) | 4 | (2) | 136 | (68) | 126 | (63) | 106 | (53) | 97 | (58) | 199 | (43) |
| Cancer | | | | | | | | | | | | | | |
| stage, n (%) | | | | | | | | | | | | | | |

| | | | | | | | | | | | | | |
|---|---|---|---|---|---|---|---|---|---|---|---|---|---|
| I | 8 | (3) | 25 | (13) | 36 | (18) | 87 | (43) | 31 | (16) | 8 | (5) | — |
| II | 27 | (11) | 91 | (45) | 70 | (35) | 29 | (15) | 60 | (30) | 65 | (39) | — |
| III | 14 | (6) | 75 | (37) | 67 | (33) | 34 | (17) | 65 | (32) | 72 | (43) | — |
| IV | 192 | (78) | 9 | (5) | 27 | (14) | 51 | (25) | 45 | (22) | 20 | (12) | — |

| | | | | | | | | | |
|---|---|---|---|---|---|---|---|---|---|
| Not staged* | 6 | (2) | — | — | — | — | — | 459 | (100) |

| | | | | | | | | |
|---|---|---|---|---|---|---|---|---|
| Unknown** | — | — | — | — | — | 1 | (1) | — |

*Does not require staging.
**Cancer stage and/or staging information has not been recorded.
**Brain cancers split by grade, as described in Table 10

TABLE 5

| | | | NCS Females Only | NCS Males Only |
|---|---|---|---|---|
| | Ovary | Prostate | (NCS-F) | (NCS-M) |
| | (n = 125) | (n = 199) | (n = 260) | (n = 199) |
| Age, years | | | | |
| Mean | 61 | 62 | 56 | 59 |
| Min-max | 21-88 | 43-87 | 20-90 | 20-90 |
| Sex, n (%) | | | | |
| Female | 125 (100) | — | 260 (100) | — |
| Male | — | 199 (100) | — | 199 (100) |
| Cancer stage, n (%) | | | | |
| I | 31 (25) | 4 (2) | — | — |
| II | 14 (11) | 160 (80) | — | — |
| III | 51 (40) | 31 (16) | — | — |
| IV | 29 (23) | 4 (2) | — | — |
| Not staged* | — | — | 260 (100) | 199 (100) |

*Does not require staging.

TABLE 6

| | Sensitivity-tuned | | Specificity-tuned | |
|---|---|---|---|---|
| Organ Specific | Sensitivity | Specificity | Sensitivity | Specificity |
| Classifier | (%) | (%) | (%) | (%) |
| Brain v NCS | 91 | 64 | 74 | 91 |
| Breast v NCS | 93 | 31 | 38 | 93 |
| Colorectal v NCS | 91 | 76 | 77 | 90 |
| Kidney v NCS | 92 | 79 | 71 | 91 |
| Lung v NCS | 93 | 78 | 69 | 90 |
| Ovarian v NCS-F | 92 | 54 | 58 | 90 |
| Pancreatic v NCS | 92 | 52 | 57 | 90 |
| Prostate v NCS-M | 92 | 51 | 66 | 93 |

TABLE 7

| | | | Identified | |
|---|---|---|---|---|
| Cancer Type | Stage | Actual | Correctly | Detection Rate (%) |
| Brain*** | I | 8 | 2 | 25 |
| | II | 27 | 4 | 15 |
| | III | 14 | 3 | 21 |
| | IV | 192 | 100 | 52 |
| | Not Staged* | 6 | 2 | 33 |
| Breast | I | 25 | 8 | 32 |
| | II | 91 | 43 | 47 |
| | III | 75 | 44 | 59 |
| | IV | 9 | 7 | 78 |
| Colorectal | I | 36 | 18 | 50 |
| | II | 70 | 25 | 36 |
| | III | 67 | 34 | 51 |
| | IV | 27 | 12 | 44 |
| Kidney | I | 87 | 40 | 46 |
| | II | 29 | 19 | 66 |
| | III | 34 | 13 | 38 |
| | IV | 51 | 15 | 29 |
| Lung | I | 31 | 11 | 35 |
| | II | 60 | 24 | 40 |
| | III | 65 | 39 | 60 |
| | V | 45 | 20 | 44 |
| Ovary | I | 31 | 12 | 39 |
| | II | 14 | 5 | 36 |
| | III | 51 | 27 | 53 |
| | IV | 29 | 13 | 45 |
| Pancreas | I | 8 | 3 | 38 |
| | II | 65 | 24 | 37 |
| | III | 72 | 32 | 44 |
| | IV | 20 | 11 | 55 |
| | Unknown** | 1 | 1 | 100 |
| Prostate | I | 4 | 1 | 25 |
| | II | 160 | 68 | 43 |
| | III | 31 | 18 | 58 |
| | IV | 4 | 1 | 25 |

*Does not require staging
**Cancer stage and/or staging information has not been recorded
***Brain cancers split by grade, as described in Table 10

TABLE 8

| | Approximate | | | |
|---|---|---|---|---|
| | wavenumber | | Biological | |
| tClassification | (cm⁻¹) | Importance | assignments | Vibrational Modes |
| C v NC | 1163 | 100 | Proteins | C—O, C—C stretching, C—H |
| | | | Carbohydrates | deformation |
| | 1578 | 99 | Amide II of Proteins | N—H bending, C—N stretching |
| | 1682 | 74 | Amide I of Proteins | C═O and C—N stretch, N—H |
| | | | | bending |
| Brain v NCS | 1595 | 100 | Amide II of Proteins | N—H bending, C—N stretching |
| | 2930 | 50 | Lipids | C—H, CH₂ stretching |
| | 1525 | 48 | Amide II of Proteins | N—H bending, C—N stretching |
| Breast v NCS | 1025 | 100 | Glycogen, | C—O and C—C stretch, C—OH |
| | | | Carbohydrates | deformation |
| | 1260 | 91 | Amide III of Proteins, | N—H in-plane bend, C—N |
| | | | Phosphodiesters | stretch, asymmetric |
| | | | | PO₂̅ stretch |
| | 1337 | 82 | Phospholipids, | CH₂ wagging, C—O stretch |
| | | | Collagen | |
| Colorectal v NCS | 1682 | 100 | Amide I of Proteins | C═O and C—N stretch, N—H |
| | | | | bending |
| | 1575 | 96 | Amide II of Proteins | N—H bending, C—N stretching |
| | 1165 | 81 | Proteins, | C—O, C—C stretching, C—H |
| | | | Carbohydrates | deformation |
| Kidney v NCS | 1682 | 100 | Amide I of Proteins | C═O and C—N stretch, N—H |
| | | | | bending |
| | 1590 | 99 | Amide II of Proteins | N—H bending, C—N stretching |
| | 1164 | 76 | Proteins, | C—O, C—C stretching, C—H |
| | | | Carbohydrates | deformation |
| Lung v NCS | 1074 | 100 | Nucleic acids, | Symmetric PO₂̅ stretch, C—O |
| | | | Phosphodiesters | stretch |
| | 1190 | 84 | Nucleic Acids | Asymmetric PO₂̅ stretch |
| | 1288 | 62 | Amide III of Proteins | N—H in-plane bend, C—N |
| | | | | stretch |
| Ovary v NCS-F | 1569 | 100 | Amide II of Proteins | N—H bending, C—N stretching |
| | 1412 | 92 | Lipids, Proteins | C—O stretch, C—H and N—H |
| | | | | deformation |
| | 1135 | 61 | Carbohydrates | C—O and C—C stretching |
| Pancreas v NCS | 1577 | 100 | Amide II of Proteins | N—H bending, C—N stretching |
| | 1074 | 86 | Nucleic acids, | Symmetric PO₂̅ stretch, C—O |
| | | | Phosphodiesters | stretch |
| | 1289 | 82 | Amide III of Proteins | N—H in-plane bend, C—N |
| | | | | stretch |
| Prostate v NCS-M | 1356 | 100 | Lipids, Proteins | C—O stretch, C—H and N—H |
| | | | | deformation |
| | 1499 | 71 | Amide II of Proteins | N—H bending, C—N stretching |
| | 1642 | 69 | Amide I of Proteins | C═O and C—N stretch, N—H |
| | | | | bending |

TABLE 9

| | Approximate | | | |
|---|---|---|---|---|
| | wavenumber | | Biological | |
| Classification | (cm⁻¹) | Importance | assignments | Vibrational Modes |
| C v NC | 1530 | 100 | Amide II of | N—H bending, C—N stretching |
| | | | Proteins | |
| | 1260 | 58 | Amide III of | N—H in-plane bend, C—N |
| | | | Proteins, | stretching, asymmetric |
| | | | Phosphodiesters | PO₂̅ stretching |
| | 1025 | 58 | Glycogen, | C—O and C—C stretching, C—OH |
| | | | Carbohydrates | deformation |
| | 1061 | 57 | Nucleic acids, | Symmetric PO₂̅ stretching, C—O |
| | | | Phosphodiesters | stretching |
| | 3345 | 56 | Amide A of | OH, C—H, N—H stretching |
| | | | Proteins | |
| Brain v NCS | 1523 | 100 | Amide Il of | N—H bending, C—N stretching |
| | | | Proteins | |
| | 1607 | 75 | Amide I of | C═O and C—N stretching, N—H |
| | | | Proteins | bending |
| | 3278 | 58 | Proteins (Amide | Symmetric O—H stretching, N—H |
| | | | A), Nucleic acids | stretching |
| | 2861 | 52 | Lipids | C—H, CH₂ stretching |
| | 1256 | 49 | Amide III of | N—H in-plane bend, C—N |
| | | | Proteins, | stretching, asymmetric |
| | | | Phosphodiesters | PO₂̅ stretching |
| Breast v NCS | 2872 | 100 | Glycogen, | C—O and C—C stretching, C—OH |
| | | | Carbohydrates | deformation |
| | 1261 | 80 | Amide III of | N—H in-plane bend, C—N |
| | | | Proteins, | stretching, asymmetric |
| | | | Phosphodiesters | PO₂̅ stretching |
| | 1549 | 76 | Amide II of | N—H bending, C—N stretching |
| | | | Proteins | |
| | 3351 | 74 | Amide A of | OH, C—H, N—H stretching |
| | | | Proteins | |
| | 1025 | 65 | Glycogen, | C—O and C—C stretching, C—OH |
| | | | Carbohydrates | deformation |
| Colorectal v NCS | 1530 | 100 | Amide II of | N—H bending, C—N stretching |
| | | | Proteins | |
| | 3351 | 69 | Amide A of | O—H, C—H, N—H stretching |
| | | | Proteins | |
| | 3234 | 65 | Proteins (Amide | Symmetric O—H stretching, N—H |
| | | | A), Nucleic acids | stretching |
| | 1246 | 59 | Amide III of | N—H in-plane bend, C—N |
| | | | Proteins, | stretching, asymmetric |
| | | | Phosphodiesters | PO₂̅ stretching |
| | 1666 | 54 | Amide I of | C═O and C—N stretching, N—H |
| | | | Proteins | bending |
| Kidney v NCS | 1258 | 100 | Amide III of | N—H in-plane bend, C—N |
| | | | Proteins, | stretching, asymmetric |
| | | | Phosphodiesters | PO₂̅ stretching |
| | 3345 | 86 | Amide A of | O—H, C—H, N—H stretching |
| | | | Proteins | |
| | 1531 | 83 | Amide II of | N—H bending, C—N stretching |
| | | | Proteins | |
| | 1348 | 65 | Lipids, Proteins | C—O stretching, C—H and N—H |
| | | | | deformation |
| | 1664 | 65 | Amide I of | C═O and C—N stretching, N—H |
| | | | Proteins | bending |
| Lung v NCS | 1167 | 100 | Nucleic Acids | Asymmetric PO₂̅ stretching |
| | 1074 | 98 | Nucleic acids, | Symmetric PO₂̅ stretching, C—O |
| | | | Phosphodiesters | stretching |
| | 1532 | 74 | Amide II of | N—H bending, C—N stretching |
| | | | Proteins | |
| | 2750 | 66 | Lipids | C—H, CH₂ stretching |
| | 1124 | 63 | Carbohydrates | C—O and C—C stretching |
| Ovary v NCS-F | 1529 | 100 | Amide Il of | N—H bending, C—N stretching |
| | | | Proteins | |
| | 3327 | 82 | Amide A of | O—H, C—H, N—H stretching |
| | | | Proteins | |
| | 3244 | 79 | Proteins (Amide | Symmetric O—H stretching, N—H |
| | | | A), Nucleic acids | stretching |
| | 1263 | 74 | Amide III of | N—H in-plane bend, C—N |
| | | | Proteins, | stretching, asymmetric |
| | | | Phosphodiesters | PO₂̅ stretching |
| | 1084 | 70 | Nucleic acids, | Symmetric PO₂̅ stretching, C—O |
| | | | Phosphodiesters | stretching |
| Pancreas v NCS | 3277 | 100 | Proteins (Amide | Symmetric O—H stretching, N—H |
| | | | A), Nucleic acids | stretching |
| | 1529 | 84 | Amide II of | N—H bending, C—N stretching |
| | | | Proteins | |
| | 1636 | 71 | Amide I of | C═O and C—N stretching, N—H |
| | | | Proteins | bending |
| | 1288 | 67 | Amide III of | N—H in-plane bend, C—N |
| | | | Proteins, | stretching, asymmetric |
| | | | Phosphodiesters | PO₂̅ stretching |
| | 2784 | 64 | Lipids | C—H, CH₂ stretching |
| Prostate v NCS-M | 1500 | 100 | Amide II of | N—H bending, C—N stretching |
| | | | Proteins | |
| | 1357 | 81 | Lipids, Proteins | C—O stretching, C—H and N—H |
| | | | | deformation |
| | 3250 | 69 | Proteins (Amide | Symmetric O—H stretching, N—H |
| | | | A), Nucleic acids | stretching |
| | 2947 | 66 | Lipids | C—H, CH₂ stretching |
| | 1003 | 64 | Glycogen, | C—O and C—C stretching, C—OH |
| | | | Carbohydrates | deformation |

TABLE 10

| WHO Grade | Brain Tumor Type | Number of patients |
|---|---|---|
| I | Meningioma | 6 |
| | Schwannoma | 1 |
| | Subependymoma | 1 |
| II | Astrocytoma | 8 |
| | Ependymoma | 1 |
| | Meningioma | 10 |
| | Oligodendroglioma | 7 |
| | Pituitary Adenoma | 1 |
| III | Astrocytoma | 7 |
| | Medulloblastoma | 1 |
| | Oligodendroglioma | 6 |
| IV | Glioblastoma | 148 |
| | Gliosarcoma | 2 |
| | Medulloblastoma | 2 |
| | Metastatic | 40 |

TABLE 11

| Model | Tuning | CI Sens (%) | CI Spec (%) |
|---|---|---|---|
| C v NCA | 98% Sens | (97.6, 98.0) | (56.9, 61.2) |
| | 98% Spec | (55.7, 57.8) | (98.1, 99.7) |
| C v NC | 90% Sens | (89.8, 90.6) | (58.8, 60.5) |
| | 95% Spec | (39.3, 40.9) | (94.3, 95.3) |
| Brain v NCS | 45% Sens | (44.6, 47.6) | (98.8, 99.3) |
| | 45% Spec | (94.7, 96.3) | (40.8, 43.9) |
| | 90% Sens | (89.0, 90.8) | (61.0, 63.8) |
| | 90% Spec | (72.4, 75.2) | (89.6, 90.7) |
| Breast v NCS | 45% Sens | (44.9, 48.7) | (85.9, 87.8) |
| | 45% Spec | (86.8, 88.9) | (42.5, 44.5) |
| | 90% Sens | (91.7, 93.6) | (30.5, 32.6) |
| | 90% Spec | (34.8, 37.5) | (90.8, 92.4) |
| Colorectal v NCS | 45% Sens | (43.3, 46.5) | (96.3, 97.2) |
| | 45% Spec | (96.6, 97.8) | (42.4, 45.5) |
| | 90% Sens | (89.5, 91.4) | (73.7, 75.8) |
| | 90% Spec | (73.4, 76.7) | (88.9, 90.5) |
| Kidney v NCS | 45% Sens | (42.9, 46.0) | (95.0, 96.1) |
| | 45% Spec | (98.6, 99.3) | (40.3, 42.5) |
| | 90% Sens | (90.4, 92.3) | (77.7, 79.4) |
| | 90% Spec | (68.7, 71.4) | (89.5, 90.9) |
| Lung v NCS | 45% Sens | (45.1, 48.6) | (94.1, 95.3) |
| | 45% Spec | (99.3, 99.8) | (38.2, 40.9) |
| | 90% Sens | (90.7, 92.8) | (76.7, 78.6) |
| | 90% Spec | (65.7, 69.2) | (88.6, 90.1) |
| Ovary v NCS-F | 45% Sens | (44.3, 48.4) | (94.7, 96.1) |
| | 45% Spec | (93.4, 95.6) | (41.0, 43.9) |
| | 90% Sens | (90.8, 93.2) | (52.0, 55.3) |
| | 90% Spec | (58.6, 62.3) | (88.5, 90.4) |
| Pancreas v NCS | 45% Sens | (41.6, 45.7) | (92.2, 93.7) |
| | 45% Spec | (94.3, 96.0) | (40.9, 43.6) |
| | 90% Sens | (91.0, 93.0) | (50.6, 53.3) |
| | 90% Spec | (51.9, 55.9) | (89.0, 90.7) |
| Prostate v NCS-M | 45% Sens | (43.5, 46.7) | (95.8, 97.2) |
| | 45% Spec | (92.3, 94.2) | (42.1, 45.6) |
| | 90% Sens | (90.3, 92.5) | (49.1, 53.0) |
| | 90% Spec | (63.2, 66.6) | (89.8, 91.8) |

### REFERENCES

1. World Health Organization, Cancer. Accessed August 2021 (2021), (available at https://www.who.int/news-room/fact-sheets/detail/cancer).
2. S. Testori, Public Health England, Cancer Research UK, What's the Most Common Treatment For Cancer? Cancer patients diagnosed at an earlier stage are more likely to have surgery than chemotherapy (2017), (available at https://news.cancerresearchuk.org/2017/10/26/cancer-patients-diagnosed-at-an-earlier-stage-are-more-likely-to-have-surgery-than-chemotherapy/).
3. M. Arruebo, N. Vilaboa, B. Seez-Gutierrez, J. Lambea, A. Tres, M. Valladares, A. González-Fernández, Assessment of the Evolution of Cancer Treatment Therapies. Cancers. 3, 3279-3330 (2011).
4. G. D. Braunstein, Cedars-Sinai Medical Center and the David Geffen School of Medicine at UCLA, Los Angeles, CA, USA, J. J. Ofman, GRAIL, Inc., Menlo Park, CA, USA, Criteria for Evaluating Multi-cancer Early Detection Tests. Oncology & Haematology. 17, 3 (2021).
5. E. Crowley, F. Di Nicolantonio, F. Loupakis, A. Bardelli, Liquid biopsy: monitoring cancer-genetics in the blood. Nat Rev Clin Oncol. 10, 472-484 (2013).
6. G. Poste, Bring on the biomarkers. Nature. 469, 156-157 (2011).
7. MDx Health, MDxHealth Select MDx Test. Accessed April 2021, (available at https://mdxhealth.com/tests/#selectMdx).
8. Exosome Diagnostics GmbH, ExoDx Test. Accessed April 2021, (available at https://www.exosomedx.com/europe/our-technology).
9. American Cancer Society, The Global Cancer Burden; Why Global Cancer Rates are Rising. Accessed November 2021, (available at https://www.cancer.org/health-care-professionals/our-global-health-work/global-cancer-burden.html).
10. H. J. Butler, P. M. Brennan, J. M. Cameron, D. Finlayson, M. G. Hegarty, M. D. Jenkinson, D. S. Palmer, B. R. Smith, M. J. Baker, Development of high-throughput ATR-FTIR technology for rapid triage of brain cancer. Nat Commun. 10, 4501 (2019).
11. J. M. Cameron, H. J. Butler, D. S. Palmer, M. J. Baker, Biofluid spectroscopic disease diagnostics: A review on the processes and spectral impact of drying. Journal of Biophotonics. 11, e201700299 (2018).
12. K. Hajian-Tilaki, Receiver Operating Characteristic (ROC) Curve Analysis for Medical Diagnostic Test Evaluation. Caspian J Intern Med. 4, 627-635 (2013).
13. G. Putcha, A. Gutierrez, S. Skates, Multicancer Screening: One Size Does Not Fit All. JCO Precision Oncology, 574-576 (2021).

1. A method of detecting whether or not a subject has a cancer irrespective of the stage or type of cancer, the method comprising:
performing an IR spectroscopic analysis comprising wavelengths between 400-4000 cm⁻¹ on a blood sample from the subject, to produce a spectroscopic signature characteristic of the blood sample, wherein said spectroscopic signature of the blood sample is analysed against representative signatures from previous subjects with and without cancer, wherein the previous subjects with cancer comprise subjects with different types of cancer and different stages of cancer, in order to detect whether or not the subject has a cancer, based upon the spectroscopic signature obtained from the subject.

2. The method according to claim 1, wherein the IR spectroscopic analysis comprises ATR-IR spectroscopic analysis.

3. The method according to claim 1, wherein a background spectrum is obtained in order to provide for correction for a background environment.

4. The method according to claim 1, wherein the spectroscopic analysis further comprises normalisation, noise reduction and/or derivatisation as one or more pre-processing step(s) and/or Fourier transform IR (FTIR) spectroscopic analysis.

5. The method according to claim 1, wherein the IR spectroscopic analysis of the blood sample further comprises detection of the type of cancer in subjects with cancer based upon the spectroscopic signature obtained from the subject.

6. The method according to claim 1, wherein the analysis of the spectroscopic signature may be used to provide an indication of brain cancer, breast cancer, colorectal cancer, kidney cancer, lung cancer, ovarian cancer, pancreatic cancer and/or prostate cancer.

7. The method according to claim 1, wherein the analysing against representative signatures from previous subjects comprises applying a trained model to the spectroscopic signature.

8. The method according to claim 7, wherein the trained model comprises a trained machine learning model, optionally a neural network, a support vector machine (SVM), a random forest (RF) decision tree, an ordered random forest (ORF) model.

9. The method according to claim 7, wherein the trained model comprises or functions as a classifier by applying the or a probability threshold to a probability value output by the trained model.

10. The method according to claim 9, further comprising selecting and/or varying the probability threshold thereby selecting and/or varying the specificity and/or sensitivity of the spectroscopic signature analysis.

11. The method according to claim 9, further comprising selecting the probability threshold based on a receiver operating characteristic (ROC) curve for the trained model, and/or selecting the trained model from a set of trained models based on ROCs for the set of trained models, for example thereby to obtain a desired specificity and/or sensitivity.

12. The method according to claim 1, wherein the analysis comprises (i) detecting whether or not the subject has cancer and/or (ii) detection of the type of cancer in the subject.

13. The method according to claim 1, wherein the spectroscopic analysis comprises detection of vibrational mode(s) and/or wavenumber regions.

14. The method according to claim 12 for detecting whether or not the subject has cancer and if the subject has cancer, the type of cancer, is based on spectroscopic signatures comprising one or more molecular vibrational mode information, wherein the molecular vibrational mode is selected from: N—H (in-plane) bend/deformation, C—N stretch, C—H stretch/deformation, CH₂ stretch, C—O stretch, C—C stretch, C—OH deformation, asymmetric/symmetric PO₂⁻ stretch, CH₂ wagging and/or C═O stretch.

15. (canceled)

16. The method of claim 1 for detecting whether or not the subject has cancer and if the subject has cancer, the type of cancer, is based on the identification of peaks in the spectroscopic signature of the sample at one or more wavenumber regions within 400-4000 cm⁻¹.

17. The method of claim 1, wherein the method is conducted in combination with other blood-based tests using a single blood draw, wherein a first aliquot is used to conduct the ATR-IR spectroscopic analysis and a second aliquot comprising the remainder of the blood sample, or a portion thereof, is used to conduct other blood-based tests.

18. A computer program product comprising computer readable instructions that are executable to perform a method according to claim 1.

19. A trained model configured to receive an input comprising a spectroscopic signature of an ATR-IR spectroscopic analysis comprising wavelengths between 400-4000 cm⁻¹ performed on a blood sample from the subject and to provide an output representative of whether or not the subject has a cancer, irrespective of the stage or type of cancer.

20. A method of training a model comprising:
receiving a plurality of data sets representing spectroscopic signatures for a plurality of subjects obtained from ATR-IR spectroscopic analysis comprising wavelengths between 400-4000 cm⁻¹ on blood samples from the subjects;
receiving cancer data indicating for at least some of the subjects whether the subject has cancer; and
training the model to determine a probability of whether a patient has cancer based on a spectroscopic signature for the patient, wherein
some of the subjects have cancer and the subjects with cancer comprise subjects with different types of cancer and different stages of cancer.

21. A method according to claim 20, wherein the training of the model comprises tuning at least one parameter of the model to optimise the area under the ROC curve and/or to provide a desired sensitivity and/or specificity for a determination as to whether a patient has cancer.
